(19) United States
(12) Patent Application Publication
(10) Pub. No.: US 2015/0031092 A1
(43) Pub. Date: Jan. 29, 2015

(54) PROCESS FOR PRETREATMENT OF THE LIGNOCELLULOSIC BIOMASS WITH A HYDRATED INORGANIC SALT MAKING IT POSSIBLE TO OBTAIN A CELLULOSIC FRACTION AND A HEMICELLULOSIC FRACTION

(71) Applicant: IFP ENERGIES NOUVELLES, RUEIL-MALMAISON CEDEX (FR)

(72) Inventors: Christophe Vallee, Sassenage (FR); Didier Bernard, Lyon (FR); Caroline Aymard, Lyon (FR); Helene Olivier-Bourbigou, Saint Genis-Laval (FR)

(73) Assignee: IFP ENERGIES NOUVELLES, RUEIL-MALMAISON CEDEX (FR)

(21) Appl. No.: 14/372,617

(22) PCT Filed: Dec. 11, 2012
(86) PCT No.: PCT/FR2012/000514

§ 371 Date: Jul. 16, 2014

(30) Foreign Application Priority Data
Jan. 18, 2012 (FR) 12/00156

(51) Int. Cl. C12P 19/14; C12P 19/02

(52) U.S. Cl. CPC C12P 19/14; C12P 19/02
USPC 435/99

(57) ABSTRACT

A method for pretreating lignocellulosic biomass by: a) acid hydrolysis with an acid solution resulting in a liquid fraction and a solid fraction, b) separating the solid fraction and the liquid fraction, c) drying the solid fraction, d) firing the dried solid fraction in a medium comprising at least one hydrated inorganic salt to obtain a solid fraction and a liquid fraction, e) separating the solid fraction and the liquid fraction obtained in d), f) optionally, treating said solid fraction obtained in e), g) enzymatic hydrolysis of said solid fraction obtained in e) and/or f), and wherein at least a portion of the liquid fraction obtained in b) is used to grow microorganisms which produce the enzymes necessary for the enzymatic hydrolysis of g).

### FIELD OF THE INVENTION

This invention is part of the framework of the processes for pretreatment of the lignocellulosic biomass. More specifically, it is part of the framework of a process for pretreatment of the lignocellulosic biomass for the production of so-called “second-generation” alcohol.

### PRIOR ART

Owing to the increase in pollution and global warming, many studies are currently being conducted to use and optimize the renewable bioresources, such as lignocellulosic biomass.

Lignocellulosic biomass consists of three primary components: cellulose (35 to 50%), hemicellulose (23 to 32%), which is a polysaccharide that essentially consists of pentoses and hexoses, and lignin (15 to 25%), which is a macromolecule with a complex structure and high molecular weight, originating from the copolymerization of phenylpropenoic alcohols. These different molecules are responsible for the inherent properties of the plant wall and are organized in a complex intertwining.

Cellulose, which comprises the majority of this biomass, is thus the most abundant polymer on Earth and the one that has the greatest potential for forming materials and biofuels. However, the potential of the cellulose and its derivatives has thus far not been able to be completely exploited, for the most part because of the difficulty of extracting the cellulose. Actually, this stage is made difficult by the very structure of the plants. The technological barriers identified in the extraction and in the transformation of the cellulose are in particular its accessibility, its crystallinity, its degree of polymerization, and the presence of hemicellulose and lignin. It is therefore essential to develop new methods for pretreatment of lignocellulosic biomass for easier access to cellulose and to make possible its transformation.

In particular, the production of biofuel is an application that requires a pretreatment of the biomass. Actually, as feedstock, the second generation of biofuel uses plant or agricultural waste, such as wood, straw, or plantings dedicated to high growth potential, such as miscanthus. This raw material is perceived as a permanent alternative solution that has little or no impact on the environment, and its low cost and its high level of availability make it a solid candidate for the production of biofuels.

The production of chemical intermediate compounds by biotechnological processes, which use in particular one or more fermentation stages, also requires a pretreatment of the biomass for using a lignocellulosic raw material whose use does not compete with food.

The principle of the process for conversion of the lignocellulosic biomass by biotechnological processes uses a stage for enzymatic hydrolysis of the cellulose contained in the plant materials for producing glucose. Next, the glucose that is obtained can be fermented into different products such as alcohols (ethanol, 1,3-propanediol, 1-butanol, 1,4-butanediol, . . . ) or acids (acetic acid, lactic acid, 3-hydroxypropionic acid, fumaric acid, succinic acid, . . . ). For the production of biofuels, glucose is generally fermented into ethanol.

However, the cellulose contained in the lignocellulosic biomass is particularly refractory to the enzymatic hydrolysis, in particular because the cellulose is not directly accessible to the enzymes. To be free of this refractory nature, a pretreatment stage upstream from the enzymatic hydrolysis is necessary. There are many methods for chemical, enzymatic, and microbiological treatment of cellulose-rich materials for improving the subsequent stage of enzymatic hydrolysis.

These methods are, for example: vapor explosion, the organosolv process, hydrolysis with dilute or concentrated acid, or else the AFEX (“Ammonia Fiber Explosion”) process. These techniques are still perfectible and in particular are subject to costs that are still too high, corrosion problems, low yields, and difficulties of extrapolation on the industrial level (F. Talebnia, D. Karakashev, I. Angelidaki Biores. Technol. 2010, 101, 4744-4753).

The acid hydrolysis of the hemicellulose is easier than that of the cellulose, and a hydrolysis of the hemicellulose can constitute the first stage of a treatment of the lignocellulosic biomass (P. Mäki-Arvela, T. Salmi, B. Holmborn, S. Willfor, and D. Yu Murzin, Chem. Rev. 2011, 111, 5638-5666). Consequently, the use of an acid pretreatment, such as hydrolysis by dilute or concentrated acid or else with vapor explosion, leads to the production of a pretreated substrate (solid fraction, containing in particular the cellulosic fraction) and a sweetened solution containing the sugars obtained from partial or total acid hydrolysis of hemicelluloses (in the form of soluble oligomers and/or monomers). This fractionation is advantageous to the extent that it makes it possible to upgrade separately the fractions that are obtained from cellulose and hemicellulose.

However, the pretreatments under acid conditions are penalized in terms of their tendency to form products of degradations. Among these degradation products, it is possible to cite the furfural that is obtained from the degradation of pentoses, 5-HMF, formic acid, or levulinic acid obtained from the degradation of hexoses, as well as aldehydes or phenolic alcohols obtained from acid degradations of the partially solubilized lignin. These degradation products can, based on their concentration, inhibit the fermentation organisms. The formation of these degradation products increases with the severity of the pretreatment (temperature, reaction period, acidity). Thus, an acid pretreatment carried out under severe conditions will make it possible to obtain a pretreated substrate (solid fraction containing cellulose) that has good susceptibility to enzymatic hydrolysis, but the associated liquid fraction will contain sugars obtained from the hydrolysis of “polluted” hemicelluloses by the presence of degradation products. In contrast, if the acid pretreatment is carried out under relatively benign conditions, the upgrading of the hemicellulosic fraction will not be penalized by the presence of degradation products, but the susceptibility of the solid substrate to enzymatic hydrolysis will be mediocre.

The applications WO2011/027220 and WO2011/027223 describe a process for pretreatment of the lignocellulosic biomass using hydrated inorganic salts preceded by a demineralization stage with an aqueous solution of acid or base. These applications do not focus on an upgrading of hemicellulose.

A process that makes it possible to transform the lignocellulosic biomass into fermentable sugars with excellent yields was recently described in the applications FR10/03092, FR10/03093 and FR11/02730 of the applicant. This process uses baking of the biomass in hydrated inorganic salts that are reactive, inexpensive, widely available, and recyclable. This technology is simple to use and makes it possible to easily consider an extrapolation on the industrial level.

However, the compositional analyses carried out on the solid fraction obtained from this pretreatment show that the hemicellulose contained in the biomass is hydrolyzed during the baking. The products resulting from this hydrolysis are therefore found in the liquid fraction that constitutes the anti-solvent and hydrated inorganic salt. The upgrading of these hydrolysis products of hemicellulose proves difficult because of the strong salt concentration of this solution and requires a complex and cumbersome process. Furthermore, the recycling of the inorganic salt is made more complex and requires a high purging rate so as to limit the accumulation of hydrolysis products of the hemicellulose during recycling.

The object of this invention is to propose a process for pretreatment making possible an optimized upgrading of the cellulosic and hemicellulosic fractions. The pretreatment process according to the invention consists in combining an acid hydrolysis under relatively benign conditions with a pretreatment by the hydrated inorganic salts and in upgrading the sugars obtained from the acid hydrolysis for the growth of microorganisms that produce enzymes for the enzymatic hydrolysis.

### DESCRIPTION OF FIGURES

FIG. 1 is a diagrammatic representation of the process according to the invention comprising an acid hydrolysis stage, a separation stage, a drying stage, a stage for baking the dried solid fraction, a stage for separating the solid fraction, a stage for treating said solid fraction, and an enzymatic hydrolysis stage.

### DETAILED DESCRIPTION OF THE INVENTION

The process for pretreatment of the lignocellulosic biomass according to this invention comprises the following stages:
a) A stage for acid hydrolysis of the biomass by an acid solution leading to a liquid fraction that contains the bulk of the hemicellulose in the form of products of hydrolysis and acid, and a solid fraction containing the bulk of the cellulose and the lignin,
b) A stage for separating the solid fraction and the liquid fraction obtained in stage a),
c) A stage for drying the solid fraction obtained in stage b),
d) A stage for baking the dried solid fraction obtained in stage c) in the presence of or in the absence of an organic solvent, in a medium comprising at least one hydrated inorganic salt of Formula (I):

MX_{n′}n′H₂O
in which
M is a metal selected from groups 1 to 13 of the periodic table,
X is an anion, and
n is an integer between 1 and 6, and with
n′ being between 0.5 and 12,
making it possible to obtain a solid fraction and a liquid fraction containing the hydrated inorganic salt,
e) A stage for separating the solid fraction and the liquid fraction obtained in stage d),
f) Optionally, a stage for treatment of the solid fraction obtained in stage e),
g) A stage for enzymatic hydrolysis of said solid fraction obtained in stage e) and/or f),
   and in which at least a portion of the liquid fraction obtained in stage b) is used for the growth of the microorganism that produces enzymes necessary for the enzymatic hydrolysis of stage g).

The stage for acid hydrolysis by an acid solution leads to a liquid fraction that contains the bulk of the hemicellulose in the form of hydrolysis products and acid and to a solid fraction that contains the bulk of the cellulose and the lignin. This stage thus makes it possible to solubilize selectively the hemicellulose contained in the cellulosic biomass.

The process according to this invention makes it possible to recover with a good yield the sugars obtained from the hemicellulosic fraction of the biomass. The use of conditions that are relatively benign for the acid hydrolysis makes it possible to minimize the formation of degradation products of the sugars. The liquid fraction that contains the sugars obtained from the hemicellulosic fraction of the biomass therefore does not have an inhibiting effect for an upgrading by a biotechnological process, in particular for its use for the growth of the microorganism that produces enzymes that are necessary for the enzymatic hydrolysis of stage g). This fraction can be used in addition in other biotechnological processes described below.

Next, the solid fraction that contains the bulk of the cellulose and the lignin is separated from the liquid fraction. It should be noted that the cellulose contained in the solid fraction after the acid hydrolysis is not reactive in enzymatic hydrolysis.

Next, the solid fraction that contains the bulk of the cellulose and the separated lignin is dried. It should be noted that the drying stage is a stage that is essential to the success of the pretreatment process. Actually, without an intermediate drying stage, the baking stage does not lead to a reactive cellulose in enzymatic hydrolysis.

Next, the stage of baking by hydrated inorganic salts is carried out on the dried solid fraction containing the bulk of the cellulose and the lignin (but without the hemicellulose that was solubilized during the acid hydrolysis stage).

This makes it possible to obtain, after a solid/liquid separation stage, a solid fraction and a liquid fraction. This solid fraction contains the bulk of the cellulose that is present in the lignocellulosic biomass. This cellulose has the property of being particularly reactive in enzymatic hydrolysis.

Because of the elimination of hemicellulose by the preliminary acid hydrolysis, the liquid fraction that is obtained after the baking stage contains the hydrated inorganic salts in suitable purity. Actually, the liquid fraction that contains the hydrated inorganic salts is no longer “polluted” by the products for hydrolysis of the hemicellulose, as is the case without an acid hydrolysis stage.

This low organic content in the liquid fraction makes it possible to facilitate the recycling of salts in the baking stage and reduces the purging rate of this recycling.

According to a preferred variant, the acid solution used in the acid hydrolysis stage is chemically identical to the hydrated inorganic salt of the baking stage diluted in water. In this case, at least a portion of the liquid fraction that contains the hydrated inorganic salts obtained in the separation stage e) can be used, optionally with the addition of additional water, as an acid solution in the acid hydrolysis stage.

The process according to this invention makes it possible to transform effectively different types of native lignocellulosic biomass into pretreated biomass while retaining the bulk of the cellulose that is present in the starting substrate. In addition, it has the advantage of using reagents that are inexpensive, widely available, and recyclable, thus making it possible to obtain a low pretreatment cost. This technology is also simple to use and makes it possible to easily consider an extrapolation on the industrial level.

The lignocellulosic biomass, or lignocellulosic materials used in the process according to the invention, is obtained from wood (leafy and resinous), raw or treated, of agricultural by-products such as straw, plant fibers, forestry crops, residues of alcohologenic, sugar-producing and grain plants, residues of the papermaking industry, marine biomass (for example, cellulosic macroalgae) or products for transformations of cellulosic or lignocellulosic materials. The lignocellulosic materials can also be biopolymers and are preferably rich in cellulose.

Preferably, the lignocellulosic biomass that is used is wood, corn straw, wood pulp, miscanthus, rice straw, or cornstalks.

According to the process of this invention, the different types of lignocellulosic biomass can be used by themselves or in a mixture.

Below, the different stages of the process will be described in detail.

### Acid Hydrolysis (Stage a)

The acid hydrolysis stage makes it possible to solubilize selectively the hemicellulose that is contained in the lignocellulosic biomass.

The acid hydrolysis of hemicellulose can be catalyzed by inorganic acids or by organic acids. Among the acids that can be used for the hydrolysis of hemicellulose, it is possible to cite sulfuric acid, hydrochloric acid, nitric acid, ferric chloride, zinc chloride, phosphoric acid, formic acid, acetic acid, oxalic acid, trifluoroacetic acid, and maleic acid, by itself or in a mixture.

The concentration of the acid is generally between 0.001 mol/L and 1 mol/L. Preferably, the concentration of the acid is between 0.01 mol/L and 0.4 mol/L.

The acid hydrolysis of the hemicellulose can be carried out at a temperature of between ambient temperature and 150° C., preferably between 50° C. and 130° C.

The duration of the acid hydrolysis is between 10 minutes and 24 hours, preferably between 30 minutes and 6 hours.

The concentration by mass of the biomass (expressed in terms of dry material) in the acid hydrolysis stage is between 1% and 30%.

The acid hydrolysis of hemicellulose is carried out under so-called mild conditions, i.e., the sugars that are solubilized in the liquid fraction undergo very few degradation reactions (such as the dehydration of xylose into furfural), and this stage does not make it possible to obtain a reactive cellulose in enzymatic hydrolysis. One skilled in the art will know how to easily select the conditions of temperature, pH and reaction time to obtain an acid hydrolysis under so-called mild conditions.

Acid hydrolysis makes it possible to obtain a liquid fraction that contains the bulk of hemicellulose, in the form of hydrolysis products (sugars or oligomers of sugars), and acid, and a solid fraction that contains the bulk of the cellulose and the lignin. At least a portion of the liquid fraction is used for the growth of microorganisms that produce enzymes for enzymatic hydrolysis.

### Solid/Liquid Separation (Stage b)

At the end of acid hydrolysis, a separation of the liquid fraction and the solid fraction is carried out. This separation stage can be carried out by the usual techniques of solid-liquid separation, for example by decanting, by filtering, or by centrifuging.

### Drying (Stage c)

Next, the solid fraction that contains the bulk of the cellulose and the lignin that is separated is dried. The drying stage is a stage that is essential to the success of the pretreatment process. Actually, without an intermediate drying stage, the baking stage does not lead to a reactive cellulose in enzymatic hydrolysis.

The drying stage can be carried out by any processes that are known to one skilled in the art, such as by, for example, evaporation. The technologies that are known for drying by evaporation are, for example, the rotary kiln, the moving bed, the fluidized bed, the heated endless screw, and the contact with metal balls providing heat. These technologies can optionally use a gas that circulates in co-current or counter-current such as nitrogen or any other inert gas under the conditions of the reaction.

The drying stage is carried out at a temperature that is greater than or equal to 50° C.

At the end of the drying stage, the residual water content is less than 30%, in a preferred manner less than 20%, and in an even more preferred manner less than 10%.

### Baking in a Medium Comprising at Least One Hydrated Inorganic Salt (Stage d)

The stage for baking by hydrated inorganic salts makes it possible to obtain a solid fraction that contains the bulk of the cellulose that is present in the lignocellulosic biomass. This cellulose has the property of being particularly reactive in enzymatic hydrolysis. A liquid fraction that contains the hydrated inorganic salt(s) is also obtained.

The baking of the dried solid fraction is carried out in the presence of a hydrated inorganic salt of formula (I): MX_{n′}n′H₂O
in which
M is a metal that is selected from groups 1 to 13 of the periodic table,
X is an anion, and
n is an integer between 1 and 6, and with
n′ being between 0.5 and 12.

A mixture of hydrated inorganic salts can be used for baking the dried solid fraction.

The anion X can be a monovalent, divalent, or trivalent anion. In a preferred way, the anion X is a halide anion that is selected from among Cl⁻, F⁻, Br⁻, and I⁻, a perchlorate anion (ClO₄⁻), a thiocyanate anion (SCN⁻), a nitrate anion (NO₃⁻), a para-methylbenzene sulfonate anion (CH₃—C₆H₄—SO₃⁻), an acetate anion (CH₃COO⁻), a sulfate anion (SO₄²⁻), an oxalate anion (C₂O₄²⁻), or a phosphate anion (PO₄³⁻). In an even more preferred way, the anion X is a chloride.

In a preferred way, the metal M in formula (I) is selected from among lithium, iron, zinc, or aluminum.

In a particularly preferred way, the hydrated inorganic salt is selected from among:

LiCl.H₂O, LiCl.2H₂O, ZnCl₂.2.5H₂O, ZnCl₂.4H₂O and FeCl₃.6H₂O.

In a preferred way, the baking temperature is between −20° C. and 250° C., preferably between 20 and 160° C.

When the metal M of the hydrated inorganic salt is selected from the groups 1 and 2 of the periodic table, the baking temperature is preferably between 100° C. and 160° C.

When the metal M of the hydrated inorganic salt is selected from the groups 3 to 13 of the periodic table, the baking temperature is preferably between 20° C. and 100° C.

The baking period is between 0.5 minute and 168 hours, preferably between 5 minutes and 24 hours, and even more preferably between 20 minutes and 12 hours.

According to the process of this invention, several successive baking stages can be carried out.

The baking stage can be carried out in the presence of one or more organic solvents, selected from among the alcohols such as methanol, ethanol, n-propanol, isopropanol, n-butanol, isobutanol, or tert-butanol; the diols and polyols such as ethanediol, propanediol or glycerol; the amino alcohols such as ethanolamine, diethanolamine or triethanolamine; ketones such as acetone or methyl ethyl ketone; carboxylic acids such as formic acid or acetic acid, dimethylformamide, dimethylacetamide, dimethyl sulfoxide, acetonitrile; and the aromatic solvents such as benzene, toluene, xylenes, and alkanes.

According to another embodiment, the baking stage can be carried out in the absence of organic solvent.

In the baking stage, the dried solid fraction is present in a quantity of between 4% and 40% by weight of a dry mass base of the total mass of the solid fraction/hydrated inorganic salt mixture, preferably in a quantity of between 5% and 30% by weight.

### Liquid/Solid Separation (Stage e)

At the end of the baking stage, a mixture of a solid fraction containing the pretreated cellulosic substrate and a liquid fraction containing the hydrated inorganic salt or salts and optionally an organic solvent are obtained. This mixture is sent into a stage for solid/liquid separation.

This separation can be carried out directly on the mixture that is obtained from the baking stage or after at least one anti-solvent promoting the precipitation of the solid fraction is added.

In a preferred manner, the separation is carried out after at least one anti-solvent that promotes the precipitation of the solid fraction is added.

The separation of a solid fraction and a liquid fraction containing the hydrated inorganic salt and optionally anti-solvent can be carried out by the usual solid-liquid separation techniques, for example by decanting, by filtering, or by centrifuging.

The anti-solvent that is used is a solvent or a solvent mixture that is selected from among water, alcohols such as methanol, ethanol, n-propanol, isopropanol, n-butanol, isobutanol or tert-butanol; diols and polyols such as ethanediol, propanediol or glycerol; amino alcohols such as ethanolamine, diethanolamine or triethanolamine; ketones such as acetone or methyl ethyl ketone; carboxylic acids such as formic acid or acetic acid; esters such as ethyl acetate or isopropyl acetate, dimethylformamide, dimethylacetamide, dimethyl sulfoxide, and acetonitrile.

Preferably, the anti-solvent is selected from among water, methanol or ethanol.

In a very preferred manner, the anti-solvent is water by itself or in a mixture, and preferably by itself.

At the end of the separation stage e), a so-called solid fraction and a liquid fraction containing the hydrated inorganic salt or salts are obtained.

The solid fraction consists of solid material, between 5% and 60%, and preferably between 15% and 45%, and a liquid phase. The presence of liquid in this fraction is connected to the limitations of the devices for liquid/solid separation. The solid material contains the bulk of the cellulose of the initial substrate, between 60% and 100%, and preferably between 75% and 99% of the cellulose initially introduced.

The liquid fraction contains the hydrated inorganic salt or salts used during the baking stage and optionally anti-solvent. Because of the elimination of the hemicellulose by acid hydrolysis, this fraction contains only very little hemicellulose (or products derived from hemicellulose). It can contain lignin.

This low organic content in the liquid fraction makes it possible to facilitate the recycling of salts in the baking stage and reduces the purging rate of this recycling.

### Additional Treatments (Stage f)

The solid fraction that is obtained in stage e) can optionally be subjected to additional treatments (stage f). These additional treatments in particular can have as their objective to eliminate traces of hydrated inorganic salts in this solid fraction.

Stage f) for treatment of the solid fraction obtained in stage e) can be carried out by one or more washing cycles, neutralization, pressing, and/or drying.

The washing cycles can be carried out with anti-solvent or with water. The washing cycles can also be carried out with a stream originating from a unit for transformation of products obtained from the process for pretreatment of this invention.

By way of example, when the process according to this invention is used as pretreatment upstream from a unit for production of cellulosic ethanol, the washing cycles can be carried out with a stream that originates from this unit for production of cellulosic ethanol.

Neutralization can be carried out by suspending in water the solid fraction obtained in stage e) and by the addition of a base. By the term base, we refer to any chemical radical that, when it is added to water, provides an aqueous solution with a pH of greater than 7. Neutralization can be carried out by an organic or inorganic base. Among the bases that can be used for neutralization, it is possible to cite soda, potash, and ammonia.

The solid fraction that is obtained at the end of the separation stage optionally can be dried or pressed for increasing the percentage of dry material contained in the solid.

### Enzymatic Hydrolysis (Stage g)

Next, the solid fraction that is treated is sent into a stage for enzymatic hydrolysis for converting the polysaccharides into monosaccharides.

The enzymatic hydrolysis stage itself is carried out under mild conditions, at a temperature of between 40 and 60° C., preferably between 45 and 50° C., and a pH of 4.5-5.5, and preferably between 4.8 and 5.2. It is carried out by means of enzymes produced by a microorganism. The enzymatic solution added to the pretreated substrate contains enzymes that decompose the cellulose into glucose.

Microorganisms, such as fungi that belong to the genera Trichoderma, Aspergillus, Penicillium or Schizophyllum, or the anaerobic bacteria that belong to, for example, the genus Clostridium, produce these enzymes, containing in particular the cellulases and hemicellulases that are suitable for the intense hydrolysis of the cellulose and hemicelluloses.

In a very preferred way, the microorganism that is used is Trichoderma reesei.

The thus obtained monosaccharides can be transformed by fermentation. The fermentation products can be alcohols (ethanol, 1,3-propanediol, 1-butanol, 1,4-butanediol, . . . ) or acids (acetic acid, lactic acid, 3-hydroxypropionic acid, fumaric acid, succinic acid, . . . ) or any other fermentation product. For example, the monosaccharides can be easily transformed into alcohol by fermentation with yeasts such as, for example, Saccharomyces cerevisiae. Next, the fermentation must that is obtained is distilled for separating the vinasses and the alcohol that is produced. This distillation stage can be thermally integrated with the drying stage c) and/or with the purification stage h) of the inorganic salt described below.

It is possible to carry out concomitantly the enzymatic hydrolysis and the fermentation according to what is commonly called SSF (Simultaneous Saccharification and Fermentation).

### Upgrading of the Liquid Fraction Obtained in Stage b)

At least a portion of the liquid fraction obtained in stage b) is used for the growth of the microorganism that produces enzymes that are necessary for the enzymatic hydrolysis of stage g). In a preferred manner, the liquid fraction that is obtained in stage b) is neutralized before its use for the growth of the microorganism. This neutralization can be carried out by the addition of an organic base or an inorganic base. Among the bases that can be used for neutralization, it is possible to cite soda, potash, and ammonia.

The use of relatively benign conditions for the acid hydrolysis makes it possible to minimize the formation of inhibiting products. The liquid fraction that contains sugars obtained from the hemicellulosic fraction of the biomass therefore does not have an inhibiting effect for such an upgrading.

The strains used for the production of cellulolytic and/or hemicellulolytic enzymes are strains of fungi belonging to the genera Trichoderma, Aspergillus, Penicillium, Fusarium, Chrysosporium or Schizophyllum, preferably belonging to the radical Trichoderma reesei. The presence of an inductor substrate is essential to the expression of cellulolytic and/or hemicellulolytic enzymes. The highest-performing industrial strains are the strains that belong to the radical Trichoderma reesei, modified for improving the cellulolytic and/or hemicellulolytic enzymes by mutation-selection processes, such as, for example, the strain IFP CL847 (FR2555803); the strains improved by the genetic recombination techniques can also be used.

These strains are cultivated in fermenters stirred and aerated under conditions compatible with their growth and the production of enzymes. Depending on the nature, the carbon-containing substrate that is selected for obtaining the biomass is introduced into the fermenter before sterilization or is sterilized separately and introduced into the fermenter after sterilization of the latter for having an initial concentration of 20 to 35 g/L; the inductive source cannot be added in this phase. An aqueous solution that contains the substrate that is selected for the production of enzymes is prepared, preferably at a concentration of 200 to 250 g/L; this solution is to contain the inductive substrate. After the initial substrate is used up, it is injected in such a way as to provide an optimized quantity, for example between 35 and 45 mg/g of cells (“fed batch”), for Trichoderma reesei. The residual sugar concentration in the culture medium is preferably less than 1 g/L during this “fed batch” phase in such a way as to promote the production of enzymes.

The liquid fraction obtained in stage b) containing sugars can be upgraded in addition in other biotechnological sugar conversion processes. In a preferred manner, the liquid fraction that is obtained in stage b) is neutralized before its use in other biotechnological sugar conversion processes.

Any sugar conversion process that uses a living microorganism or an agent that is obtained from a living microorganism is called a biotechnological sugar conversion process for the conversion of these sugars into products of interest, and, for example:
Fermentation into ethanol of C6 sugars by a yeast, for example a yeast that belongs to the genus Saccharomyyces (S. cerevisiae, S. carlsbergensis, S. bayanus, S. uvarum), Schizosaccharomyces (S. pombe) or else Kluyveromyces (K. fragilis);
Fermentation into solvents such as acetone, butanol, by a bacterium, such as, for example, that of the genus Clostridium,
Fermentation into ethanol of C6 and C5 sugars by a yeast such as, for example, Pichia Stipitis or Candida Sheatae or Pachysolen tannophilus; or by a bacterium such as, for example, Zymomonas mobilis; or else by a genetically modified yeast for using C5;
Production of filamentous fungi, for example, T. reesei.

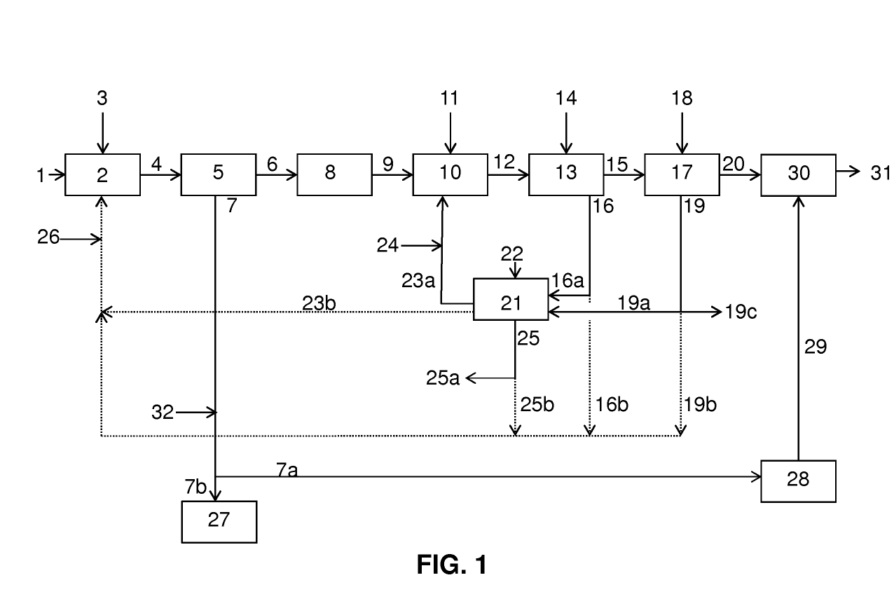

The process will be described by referring to FIG. 1.

The lignocellulosic biomass is introduced via the pipe 1 into the reactor 2 in which the acid hydrolysis stage takes place. The acid solution is introduced via the pipe 3. At the end of the acid hydrolysis stage, a mixture of a liquid fraction containing the bulk of the hemicellulose in the form of hydrolysis products (sugars or sugar oligomers) and acid and a solid fraction containing the bulk of the cellulose and the lignin is drawn off via pipe 4. This mixture is sent into the liquid/solid separation device 5 in which the separation stage b) takes place. At the end of the separation stage b), a so-called solid fraction 6 and a liquid fraction 7 are obtained.

Next, the solid fraction 6 is sent into a drying stage 8.

Next, the dried solid fraction is introduced via the pipe 9 into the baking reactor 10 in which the baking stage takes place. The baking medium comprising one or more hydrated inorganic salts and optionally an organic solvent is introduced via the pipe 11.

At the end of the baking stage, a mixture containing the pretreated lignocellulosic substrate, the hydrated inorganic salt or salts, and optionally an organic solvent is drawn off via the pipe 12. This mixture is sent into the device 13 for liquid/solid separation in which the separation stage e) takes place. The optional anti-solvent is added via the pipe 14.

At the end of the separation stage e), a so-called solid fraction 15 and a liquid fraction 16 containing the hydrated inorganic salt or salts are obtained.

The solid fraction (15) can optionally be subjected to additional treatments (stage f) carried out in the device (17). The agents that are optionally necessary for treatments(s) carried out in the chamber 17 are introduced via the pipe 18.

The optional residues of this (these) treatment(s) are drawn off via the pipe 19. The treated solid fraction is drawn off via the pipe 20 and is sent into an enzymatic hydrolysis stage in the reactor 30 for converting the polysaccharides into monosaccharides 31.

The liquid fraction 7 that is obtained in the separation stage b) and that contains the sugars that are obtained from the hemicellulose is at least in part (7a) sent into a chamber 28 for the growth of the microorganism that produces enzymes necessary for the enzymatic hydrolysis in the chamber 30. Preferably, the liquid fraction 7 is neutralized in advance by the injection of a base 32.

The thus produced enzymes are introduced via the pipe 29 into the chamber 30.

According to a variant, another portion (7b) of the liquid fraction 7 can be used in other processes 27 using a living microorganism or an agent that is obtained from a living microorganism for the conversion of these sugars into products of interest.

According to an embodiment, not shown, the separation stage e) is carried out with the addition of an anti-solvent, and the additional treatment that is carried out in the chamber 17 (stage f) consists of one or more washing cycles carried out with the anti-solvent that is introduced via the pipe 18. The liquid after washing primarily contains the anti-solvent and contains hydrated inorganic salt. This effluent is used in the separation stage e). This embodiment makes possible a better rate of recovery of the hydrated inorganic salt, a better purity of the solid fraction 20, while limiting the consumption of the anti-solvent. In a preferred manner in this embodiment, the anti-solvent is water.

According to a preferred embodiment, the inorganic salt that is contained in different liquid fractions obtained during the process can be recycled.

According to a first variant, at least a portion of the liquid fraction obtained in stage e) (16a) is sent to a purification stage (21), referred to as stage h), making it possible to concentrate the inorganic salt that is contained in the liquid fraction and to obtain a liquid fraction containing the concentrated inorganic salt (23a) and another liquid fraction that is low in inorganic salt (25), with said liquid fraction containing the concentrated inorganic salt (23a) next being recycled at least in part in the baking stage d).

The purification stage h) in particular can be a stage for separation of the inorganic salt and the anti-solvent. This separation can be carried out by any processes that are known to one skilled in the art, such as, for example, evaporation, precipitation, extraction, running the material over ion exchange resin, electrodialysis, chromatographic methods, solidification of the hydrated inorganic salt by lowering the temperature or the addition of a third body, reverse osmosis.

The additives that are optionally necessary to this stage are introduced via the pipe (22) into the chamber (21).

At the outlet of the chamber (21), a liquid fraction that contains the concentrated inorganic salt (23a) that is advantageously recycled at least in part to the baking reactor (10) (stage d) is obtained. Optionally, water can be added to the stream (23a) via the pipe (24) to adjust the stoichiometry of water and to obtain a hydrated inorganic salt with a composition that is identical to the one introduced via the pipe (11). In a preferred manner, the concentrated inorganic salt that is obtained has the same composition as the one introduced via the pipe (11). Optionally, the liquid fraction (23a) can contain all or part of the organic solvent.

The liquid fraction that is low in inorganic salt (25) can contain anti-solvent, organic solvent, residues of products derived from the biomass, and inorganic salt. In a preferred manner, the liquid fraction that is low in inorganic salt (25) contains less than 50% of the hydrated inorganic salt that is initially contained in the fraction (16). In an even more preferred manner, the liquid fraction that is low in inorganic salt (25) contains less than 25% of the hydrated inorganic salt initially contained in the fraction (16).

The liquid fraction that is low in inorganic salt (25) obtained in the chamber (21) can also be a partial purging (25a).

When stage e) is carried out with the addition of an anti-solvent, the anti-solvent is recovered for the most part in the liquid fraction that is low in inorganic salt (25) and can be recycled (not shown) to stage e) after optional retreatment or to stage f).

According to another variant, stage f) for treatment of the solid fraction obtained in stage e) is carried out by one or more washing cycles making it possible to obtain a treated solid fraction (20) and a liquid fraction (19), with said liquid fraction being at least in part (19a) sent to a purification stage (21) making it possible to concentrate the inorganic salt contained in the liquid fraction and to obtain a liquid fraction containing the concentrated inorganic salt (23a) and another liquid fraction that is low in inorganic salt (25), with said liquid fraction containing the concentrated inorganic salt (23a) next being at least in part recycled in the baking stage d).

When stage f) is carried out with the addition of an anti-solvent, the optional residues of this (these) treatment(s) are drawn off via the pipe (19) and then either purged (19c) or sent into the chamber (21) via the pipe (19a).

According to an embodiment (not shown), the anti-solvent (18) added to stage f) is separated during the purification stage (25) and recycled in stage f).

By the acid hydrolysis stage, the process according to the invention makes it possible to separate selectively the hemicellulose that has as a consequence a significant lowering of products derived from the biomass in the liquid fraction obtained after the baking stage d). According to an embodiment, not shown, and when the latter proves necessary, the very small quantity of products derived from the biomass still contained in the liquid fraction can be separated before or after the separation of the hydrated inorganic salt and the anti-solvent. The products derived from the biomass can be, for example, extracted by addition of a non-miscible solvent with the hydrated inorganic salt or with the mixture of hydrated inorganic salt and anti-solvent. The products derived from the biomass can also be precipitated by modification of conditions (temperature, pH, etc.) or by the addition of a third body. The products derived from the biomass can also be adsorbed on a solid.

### Special Case: Chemical Identity of the Acid Solution and the Hydrated Inorganic Salt Diluted in Water

According to a preferred variant, the acid solution used in stage a) is chemically identical to the hydrated inorganic salt of formula (I) of stage d) that is diluted in water.

In this case, the inorganic salt is preferably selected from among ferric chloride and/or zinc chloride, and the acid solution used for stage a) is an aqueous solution that is diluted with ferric chloride and/or zinc chloride.

The liquid fraction after the baking stage is, thanks to the preliminary acid hydrolysis stage, highly concentrated with hydrated inorganic salts without being enriched by a significant portion of the products for hydrolysis of hemicellulose. When the salt that is diluted in water and the acid solution are chemically identical, the recycling of this composition in each of the stages (acid hydrolysis and baking) then becomes possible. In addition, this makes it possible to obtain a still lower pretreatment cost because it uses a single chemical compound in the two pretreatment stages.

In this case, at least a portion of the acid solution that is used in stage a) is obtained from at least a portion of the liquid fraction that is obtained in stage e) and/or in stage f), with or without passage in a purification stage making it possible to concentrate the inorganic salt that is contained in the liquid fraction(s) and to obtain a liquid fraction that contains the concentrated inorganic salt and another liquid fraction that is low in inorganic salt and/or also obtained from at least a portion of the liquid fraction that is low in inorganic salt and that is obtained from the purification stage h).

This case is shown in FIG. 1 by arrows in dotted form. By referring to FIG. 1, a portion of the liquid fraction (16b) obtained in stage e) can be recycled (without a purification stage) in the acid hydrolysis chamber (2). Another portion of the liquid fraction (16a) can be sent to a purification stage used in the chamber (21) as described above. At the outlet of the chamber (21), a liquid fraction containing the concentrated inorganic salt (23a) and (23b) and another liquid fraction that is low in inorganic salt (25) are obtained, with a portion of said liquid fraction containing the concentrated inorganic salt (23b) next being able to be recycled in the acid hydrolysis (2), another portion of said liquid fraction containing the concentrated inorganic salt (23a) next being able to be recycled in the baking stage d).

Optionally, water can be added to the stream (23b) via the pipe (26) for adjusting the quantity of water and for obtaining an acid solution with a composition that is identical to the one introduced via the pipe (3).

According to another embodiment, not shown, another portion of the liquid fraction (16) obtained in stage e) can be recycled directly (without a purification stage) in the baking stage d).

In the same manner, at least one portion of the liquid fraction (19b) obtained in stage f) can be recycled (without a purification stage) in the acid hydrolysis chamber (2). Another portion of the liquid fraction (19a) can be sent to a purification stage used in the chamber (21) as described above. At the outlet of the chamber (21), a liquid fraction is obtained that contains concentrated inorganic salt (23a) and (23b) and another liquid fraction that is low in inorganic salt (25), a portion of said liquid fraction containing the concentrated inorganic salt (23b) next being able to be recycled in the acid hydrolysis (2), and another portion of said liquid fraction containing the concentrated inorganic salt (23a) next being able to be recycled in the baking stage d).

In the acid hydrolysis stage, it is also possible to recycle at least a portion of the liquid fraction that is low in inorganic salt (25b), obtained from the purification stage h).

1. Process for pretreatment of the lignocellulosic biomass comprising the following stages:
a) A stage for acid hydrolysis of the biomass by an acid solution leading to a liquid fraction that contains the bulk of the hemicellulose in the form of products of hydrolysis and acid and to a solid fraction containing the bulk of the cellulose and the lignin,
b) A stage for separating the solid fraction and the liquid fraction obtained in stage a),
c) A stage for drying the solid fraction obtained in stage b),
d) A stage for baking the dried solid fraction obtained in stage c) in the presence of or in the absence of an organic solvent, in a medium comprising at least one hydrated inorganic salt of Formula (I):
MX_{n′}n′H₂O
in which
M is a metal selected from the groups 1 to 13 of the periodic table,
X is an anion, and
n is an integer between 1 and 6, and with
n′ being between 0.5 and 12,
making it possible to obtain a solid fraction and a liquid fraction containing the hydrated inorganic salt,
e) A stage for separating the solid fraction and the liquid fraction obtained in stage d),
f) Optionally, a stage for treatment of the solid fraction obtained in stage e),
g) A stage for enzymatic hydrolysis of said solid fraction obtained from stage e) and/or f),
and in which at least a portion of the liquid fraction obtained in stage b) is used for the growth of the microorganism that produces enzymes necessary for the enzymatic hydrolysis of stage g).

2. Process according to claim 1, in which the acid of stage a) is selected from among sulfuric acid, hydrochloric acid, nitric acid, ferric chloride, zinc chloride, phosphoric acid, formic acid, acetic acid, oxalic acid, trifluoroacetic acid, and maleic acid, by itself or in a mixture.

3. Process according to claim 1, in which the concentration of acid of stage a) is between 0.001 mol/L and 1 mol/L.

4. Process according to claim 1, in which stage a) is carried out between ambient temperature and 150° C.

5. Process according to claim 1, in which the drying stage is carried out at a temperature that is greater than or equal to 50° C.

6. Process according to claim 1, in which the anion X of the hydrated inorganic salt of Formula (I) is a halide anion, selected from among Cl⁻, F⁻, Br⁻, I⁻, a perchlorate anion, a thiocyanate anion, a nitrate anion, an acetate anion, a para-methylbenzene sulfonate anion, a sulfate anion, an oxalate anion, or a phosphate anion, and in which the metal M in Formula (I) is selected from among lithium, iron, zinc, or aluminum.

7. Process according to claim 1, in which the baking stage d) is carried out at a temperature of between −20 and 250° C., preferably between 20 and 160° C.

8. Process according to claim 1, in which the baking stage d) is carried out in the presence of one or more organic solvents, selected from among the alcohols such as methanol, ethanol, n-propanol, isopropanol, n-butanol, isobutanol, or tert-butanol; the diols and polyols such as ethanediol, propanediol or glycerol; the amino alcohols such as ethanolamine, diethanolamine or triethanolamine; ketones such as acetone or methyl ethyl ketone; carboxylic acids such as formic acid or acetic acid, dimethylformamide, dimethylacetamide, dimethyl sulfoxide, acetonitrile; and the aromatic solvents such as benzene, toluene, xylenes, and alkanes.

9. Process according to claim 1, in which the stage e) for separating the solid fraction is carried out by precipitation by the addition of at least one anti-solvent that is a solvent or a mixture of solvents selected from among water, alcohols such as methanol, ethanol, n-propanol, isopropanol, n-butanol, isobutanol or tert-butanol; diols and polyols such as ethanediol, propanediol or glycerol; amino alcohols such as ethanolamine, diethanolamine or triethanolamine; ketones such as acetone or methyl ethyl ketone; carboxylic acids such as formic acid or acetic acid; esters such as ethyl acetate or isopropyl acetate, dimethylformamide, dimethylacetamide, dimethyl sulfoxide, and acetonitrile; and preferably, the anti-solvent is water by itself or in a mixture.

10. Process according to claim 1, in which stage f) for treatment of the solid fraction obtained in stage e) is carried out by one or more washing cycles, neutralization, pressing, and/or drying.

11. Process according to claim 1, in which the enzymatic hydrolysis g) is carried out at a temperature of between 40 and 60° C. and at a pH of between 4.5 and 5.5.

12. Process according to claim 1, in which at least a portion of the liquid fraction obtained in stage b) is neutralized before being used for the growth of the microorganism that produces enzymes.

13. Process according to claim 1, in which at least a portion of the liquid fraction obtained in stage b) is used in the biotechnological processes for conversion of sugars selected from among:
The fermentation into ethanol of C6 sugars by a yeast,
The fermentation into solvents by a bacterium,
The fermentation into ethanol of C6 and C5 sugars by a yeast, by a bacterium, or by a genetically modified yeast,
The production of filamentous fungi.

14. Process according to claim 1, in which at least a portion of the liquid fraction obtained in stage e) is sent to a purification stage, referred to as stage h), making it possible to concentrate the inorganic salt that is contained in the liquid fraction and to obtain a liquid fraction containing the concentrated inorganic salt and another liquid fraction that is low in inorganic salt, with said liquid fraction containing the concentrated inorganic salt next being at least in part recycled in the baking stage d).

15. Process according to claim 1, in which the acid solution used in stage a) is chemically identical to the hydrated inorganic salt of Formula (I) that is diluted in water.
